SEQUENCE LISTING (2 sequences)

SEQ ID NO: 1 - DNA, 4501 bp, Artificial Sequence
  Description of Artificial Sequence Synthetic polynucleotide
gattgattaatagaataatgtgacttgagaattgtcttctcaggcaaaatgctgggggaaatgcaaattgggacattcctctattcacaggtaatctgacaatatctacgaaattacagacacatttactcatcaatccagctttaggaagtctacccaaagactcacctgcgcatgtatgaaatactacatgttcaaagtcattcactgcagcctagcattgtttgctactgcaaaggactacaagcaacccaaatgcccattttatacaataaagacttgtatgataaccatgtactaacacacaaagcagctgttaaaaaaagagaacaattttataacatactgttgagttaaacagatatatacataaagaataaacttttatatttatatatcaaagtgtatatgtagccctttctctaagaaaggagggtaagaaggaaatatattcatatattccatttgtataacactggaaacaaatgaaaatggtacttataagggtgaggaggggccatgaggttgataggaaaaagggtgagagaaggtttccctttttatatgtttttctattattggatttttgaagaaagaaaatgtattaaaacaaaaaaaaattttttttatttttatcttttaacagctgtacctgggccatctggaagttcctaggctaggggccgaattagagctgcagctgccagcctacaccacggccacagcaatgacagatttgagccacagctgtgacctatgccaaagcctgctacagtgctggatccttaacccactgagtgaggccaggaattgagcccacatcctcgtggacactatattgggtttttaacatcctaaaaagttttaaaaccctatattatgtactatattcttcactaaatcagtattgtctttttactaccactttccttcttaagagtacttctataccagaaataggccaaatggtcaaacataaggggaaaatacttttcagctaattacaagaaataagatttcttgaaacaaaaaattgctgcattgaacattaatgtgtactttttaaaaagaaaatattgtctaaatcagaggttggcaatatacggtctttggaccaaacccagtcccagactatttttgtatagcctgtgagctaaggatggtttaaagggatataaagaaaaaaataaagaatatgcaagagagaccttatgcggctcacaaaaatctaaaatatttactatctggtcctccacagaaaacatttgctgacccttggctgaataatggcatcttttatatgttcttaatgaagcagcagaagtacaaactagttaccttatatgtgcagatagtcagtttcctcttcctggtttcagtgtttctctgcagtaacgtattaagcttggtaccgagctcggatccactagtaacggccgccagtgtgctggaattcggcttcaacaacttcgtataatgtatgctatacgaagttatcagtacttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcaacaacttcgtataatgtatgctatacgaagttatcagtacaagccgaattctgcagatatctcacatctggaaaacctgctgcatgactgcatcttgtctcaggtcatcacggcccttcagaataatcacaatatgaataactaagcacagttcatttcttcattgtcagatgatacagtcatctgaggacagctaaaaggcactgagaaatcaatctgagcatccatgctcctcataatagtctctcaatgaattaagacctaagatgattctttcatttagaataagctataccactgggtaggcagaccgatttagaagaaagaaggtccccctgccagctgcttttgtaattaaacccactggaactcctccacctgagggcccactgcaaacatcaggcagagcagcacctttgtctgtactactttgcagcgaaacccaaagagccggagagcagagggagggagggacgggcaaagcactcagcacagggccagacttggtccttcactccaactattcaagaaaagaagcaaccaccaaaacatggttttttcccataacaagcaggctgattgttaacctcatcatgcctgtacatgctaaaagtattctttatggggttcccactgtggcacagcaggttaaggatctggtgttgccacagctgtggtgtaggttgtagctgtggcttggacttgatccctgaccccaggaacttccatatgtcatgggtgaggcaaaaaacaaaaaaagattctttcaaccaactattataataagtctagtcttgcagagacacaaaaaaatgagaaaaagcattctgcttctagaaaataatacatttgttttcctcacctgtaattattcagaataaaaataaaagcccaaagcaagaagaaaatatcaattatagttttctatgacttctttcattaaccaactaggaaccacaccacaaaaaaaagtttcaccttgacaagctgtcgcctttccttgccatcagagccgacacaatctattatttttggtaaatttaaacctcctgctaaacgaaattctggtttaaatgactgtatagtcaccatgtttccatattctcctgtggggtccacctaataaaatgtacaaatatgacaataggtcagatacacaatttgaatttaaagaaaaatgaattataagtgacaactgtggtattaaacagaggaacgaatgacaatgtgctgttgattaagacttgaagtgataaatttataaatgaaaggaagaagatttgacattggaaaggcttttaaaaagtgacacagataacacaatttatctccctaaatatcactttaaaaaagtaggtcatacaaagttagaattacattgctaaaaaactactgaaaaacctgggtttaggtcttgccatagacctatatatggtaagtcatctgagaaatagtaagct

SEQ ID NO: 2 - DNA, 31 bp, Artificial Sequence
  Description of Artificial Sequence Synthetic oligonucleotide
aggttttccagatgtgagatatctgcagaat

(19) United States
(12) Patent Application Publication
(10) Pub. No.: US 2016/0143255 A1
(43) Pub. Date: May 26, 2016

(54) Animal Models of Ataxia-Telangiectasia (A-T)

(71) Applicant: EXEMPLAR GENETICS, LLC, Sioux Center, IA (US)

(72) Inventors: Christopher Rogers, Coralville, IA (US); John Swart, Sioux Center, IA (US)

(73) Assignee: Exemplar Genetics, LLC, Sioux Center, IA (US)

(21) Appl. No.: 14/776,677

(22) PCT Filed: Mar. 14, 2014
(86) PCT No.: PCT/US14/29248

§ 371 Date: Sep. 14, 2015

Related U.S. Application Data
(60) Provisional application No. 61/788,080, filed on Mar. 15, 2013.

(51) Int. Cl. A01K 67/027

(52) U.S. Cl. CPC A01K 67/0276; A01K 2217/075; A01K 2267/0318; A01K 2227/108

(57) ABSTRACT

The present invention provides transgenic, large non-human animal models of Ataxia-Telangiectasia, as well as methods of using such animal models in the identification and characterization of therapies for Ataxia-Telangiectasia.

### PRIORITY

This application is a §371 filing of PCT/US2014/29248, filed on Mar. 14, 2014 and claims priority to U.S. Provisional Application No. 61/788,080, filed Mar. 15, 2013, both of which are hereby incorporated by reference in their entirety.

### STATEMENT AS TO FEDERALLY SPONSORED RESEARCH

This invention was made with government support under grant number NS076075 awarded by the National Institutes of Health and the National Institute of Neurological Disorders and Stroke. The government has certain rights to this invention.

### SEQUENCE LISTING

The instant application contains a Sequence Listing which has been submitted in ASCII format via EFS-Web and is hereby incorporated by reference in its entirety. Said ASCII copy, created on Sep. 12, 2015, is named EXEM0010.txt and is 6,555 bytes in size.

### FIELD OF THE INVENTION

This invention relates to transgenic, non-human animal models of disease, cells that can be used to make such animals, and methods of using these animals and cells.

### BACKGROUND OF THE INVENTION

Many human diseases and conditions are caused by gene mutations. Substantial effort has been directed towards the creation of transgenic animal models of such diseases and conditions to facilitate the testing of approaches to treatment, as well as to gain a better understanding of disease pathology. Early transgenic animal technology focused on the mouse, while more recent efforts, which have been bolstered by the development of somatic cell nuclear transfer (SCNT), have included larger animals, including pigs, cows, and goats. This technology has resulted in the production of, for example, pigs in which the gene encoding α-1,3-galactosyltransferase has been knocked out, in efforts to generate organs that can be used in xenotransplantation (see, e.g., Lai et al., Science 295:1089-1092, 2002). Further, this technology has resulted in the production of large animal models of human cystic fibrosis (CFTR−/− and CFTR-ΔF508/ΔF508 pigs, see, e.g., U.S. Pat. No. 7,989,675 and U.S. patent application Ser. No. 12/283,980); and large animal models of human cardiovascular disease (LDLR+/− and LDLR−/− pigs, see, e.g., U.S. patent application Ser. No. 13/368,312). Additional applications of this technology include the production of large quantities of human proteins (e.g., therapeutic antibodies; see, e.g., Grosse-Hovest et al., Proc. Natl. Acad. Sci. U.S.A. 101(18): 6858-6863, 2004). Substantial benefits may be obtained by the use of somatic cell nuclear transfer technology in the production of large animal models of human disease.

One example of a condition caused in part by a genetic mutation is Ataxia-Telangiectasia (A-T) (also referred to as Louis-Bar syndrome). Ataxia refers to poor coordination and telangiectasia to small, dilated blood vessels, both of which are hallmarks of the disease.

Ataxia-Telangiectasia (A-T) is an autosomal recessive disorder with a worldwide incidence of 1 in 40,000 to 1 in 100,000 live births. Major clinical features of A-T are progressive neurodegeneration leading to cerebellar ataxia, and dilation of small blood vessels in the eyes and skin known as telangiectasia. In addition, A-T patients exhibit a number of other symptoms, including a predisposition to cancer (e.g., lymphomas, leukemia, brain tumors), hypersensitivity to ionizing radiation, immune system irregularities, chromosomal instability, insulin-resistant diabetes, and a much reduced, or absent, thymus. See, e.g., Lavin, M. F., Nat Rev Mol Cell Biol, 2008. 9(10): p. 759-69. Ataxia typically develops at an early age, with the first signs being difficulty in controlling body posture and movements. Some children with A-T may begin to walk later (after 18 months), fall frequently, and walk unsteadily or be reluctant to let go of supporting objects or people. The disease is progressive, and most patients become wheelchair-bound by their teens, followed by death in their twenties. There is currently no cure for A-T.

A-T arises from mutations in the Ataxia-Telangiectasia Mutated (ATM) gene, which was first identified and cloned in 1995 (see, e.g., Savitsky, K., et al., Science, 1995. 268(5218): p. 1749-53). The ATM gene is 160 kb in length, and encodes a transcript of 13 kb spanning 66 exons. To date, at least 432 unique mutations have been identified in ATM, the majority of which are truncating or splice-site mutations that give rise to shorter, non-functional ATM proteins. ATM is a Ser/Thr protein kinase that is a member of the phosphoinositide 3-kinase (PI3K)-related protein kinase (PIKK) family, as is Rad3-related protein (ATR), both of which are involved in DNA damage response. The kinase domain of ATM is known to act on the tumor suppressor protein p53, both in vitro and in vivo, and activation of p53 is deficient in A-T cells. See, e.g., Banin, S., et al., Science, 1998. 281(5383): p. 1674-7; Canman, C. E., et al., Science, 1998. 281(5383): p. 1677-9; Khanna, K. K., et al., Nat Genet, 1998. 20(4): p. 398-400. In addition, proteomic screens suggest that ATM and ATR may have as many as 700 substrates in vivo (Matsuoka, S., et al., Science, 2007. 316(5828): p. 1160-6). ATM also contains a number of other domains such as a focal adhesion targeting (FAT) domain, a mammalian target of rapamycin (mTOR) domain, a transformation/transcription domain-associated protein (TRRAP) domain, a Leu zipper, an N-terminal substrate binding domain, and a peroxisomal targeting signal sequence (PTSI) (Lavin, M. F., Nat Rev Mol Cell Biol, 2008. 9(10): p. 759-69).

ATM and related proteins are now known to play an important role in DNA damage repair, and that loss of ATM function results in disruptions in a number of cellular pathways. Murine models of A-T have provided insights into the consequences of ATM dysfunction but do not replicate the full repertoire of clinical symptoms observed in A-T disease. While these mice are useful for investigating some of the cellular pathways in which ATM is involved, they are not ideal for studying neurological disease associated with A-T or for testing new therapeutic approaches. A large animal model that shares anatomical, physiological, and developmental similarities with humans and more accurately models A-T disease could be a transformative resource, bridging the gap between the current mouse models and the development of effective treatments in humans. Furthermore, a large animal (for example, porcine) A-T model may serve not only as a model for A-T disease, but also as a model for other diseases such as cancer, immune disorders, and neurological disease. Therefore, a large animal A-T model would benefit multiple disciplines within research communities studying a wide variety of diseases.

Furthermore, there is great interest in advancing medical devices, interventional strategies, and non-invasive diagnostic methods beyond their current state, but these fields are also limited by the current model systems. Rodent models are not well suited for most of these applications due to their size. Therefore, in one aspect of the invention, the transgenic animal model is a new model for A-T in a miniature pig breed. In one embodiment, the present invention accomplishes this in two steps by combining gene targeting and SCNT.

### SUMMARY OF THE INVENTION

The invention provides large, non-human animal models of human diseases or conditions, in which one or more genes associated with the diseases or conditions include one or more targeted mutations. The animals of the invention can be, for example, ungulates such as pigs, cows, sheep, and goats. In one example, the disease or condition is Ataxia-Telangiectasia and the gene including one or more mutations is the Ataxia-Telangiectasia Mutated (ATM) gene.

The animal models of the invention can include the mutation(s) in one or both alleles of the ATM gene in the genome of the transgenic animal, and the mutation(s) can result in full or partial inactivation of the gene. In one example, the mutation includes an insertion of an exogenous nucleic acid molecule and/or a transcription/translation termination sequence. In another example, the mutation includes a deletion of an endogenous nucleic acid molecule or a portion thereof. In yet another example, the mutation substantially eliminates expression of a functional gene product of the targeted gene in cells in which such expression normally takes place, absent the mutation. In the case of an animal with a mutation or mutations in both alleles of a gene, the mutation or mutations in each allele can be identical to one another or can be different.

The animal models of the invention may also include a homologous transgenic copy of a wild-type or mutated gene from a different animal. In one embodiment, the invention may include an orthologous gene from a different animal. The animal models may thus include, for example, in addition to a mutation/inactivation of an endogenous gene, an inserted copy of a corresponding gene from another species. Thus, for example, an animal (such as a pig) in which an endogenous ATM gene is mutated or inactivated may be modified to include an ATM gene from another animal (such as a human), which may be wild-type or may include a mutation. The invention therefore provides transgenic, large (non-human) animal models of human diseases and conditions (e.g., pigs) in which one or more endogenous genes associated with the disease or condition are knocked-out (i.e., genetically altered in such a way as to inhibit the production or function of the product or gene) and replaced with a homologous wild-type or mutated gene derived from a different animal (e.g., a human). In one example, a pig with its endogenous porcine ATM gene knocked-out expresses a human transgene encoding the ATM gene or a mutation thereof.

The invention also provides isolated cells of transgenic, large non-human animal models of human diseases or conditions, in which one or more genes associated with the diseases or conditions include one or more targeted mutations. The animals can be, for example, ungulates, such as, e.g., pigs, cows, sheep, and goats. In one example, the disease or condition is Ataxia-Telangiectasia and the gene including one or more mutations is the ATM gene.

Examples of ATM mutations that can be included in the animals and cells of the present invention can include mutations affecting the synthesis of ATM (for example, mutations that prevent any functional ATM from being produced), and mutations that give rise to the production of an abnormal version of ATM that retains some function.

The cells of the invention can include the mutation(s) in one or both alleles of the genes in the genomes of the cells, and the mutation(s) can result in full or partial inactivation of the gene(s). In one example, the mutation includes an insertion of an exogenous nucleic acid molecule and/or a transcription/translation termination sequence. In another example, the mutation substantially eliminates expression of a functional gene product of the targeted gene in cells in which such expression normally takes place, absent the mutation. In the case of a cell with a mutation or mutations in both alleles of a gene, the mutation or mutations in each allele can be identical to one another or can be different. In one example, the cells are fetal cells, such as fetal fibroblasts. Additional examples of cell types included in the invention are provided below.

The invention further provides methods of making transgenic, large non-human animal models of diseases or conditions as described above and elsewhere herein. The methods can include the steps of: (i) introducing one or more mutations into an allele of one or more genes associated with a disease or condition in a cell (e.g., a fetal fibroblast) to generate a donor cell; (ii) introducing the nucleus of the donor cell into a recipient cell (e.g., an enucleated oocyte) to generate an embryo; and (iii) transferring the embryo into a surrogate female. The animals can be, for example, ungulates, such as, e.g., pigs, cows, sheep, and goats. In one example, the disease or condition is A-T and the gene including one or more mutations is an ATM gene. In a variation of these methods, the donor cell includes one or more mutations in one allele of an ATM gene. In another variation of these methods, the donor cell includes one or more mutations in one allele of an ATM gene, and the method is carried out to introduce one or more mutations into the other allele. In another example, the methods further involve breeding an animal that is born from the surrogate female to obtain a male mutant that exhibits symptoms of A-T.

The invention also includes methods of identifying therapeutic agents that can be used in the treatment of diseases or conditions (e.g., A-T). These methods involve administering one or more candidate therapeutic agents to a transgenic animal, as described above, and monitoring the animal for one or more symptoms of the disease or condition. Detection of improvement or other change in a symptom of the disease or condition indicates the identification of a compound that may be used in the treatment or prevention of the disease or condition.

The invention also includes methods of providing surgical training and medical imaging that can be used in the treatment of diseases or conditions (e.g., A-T). These methods involve using the transgenic animals of the present invention for the refinement of surgical techniques using standard approaches, as well as minimally invasive and robotic technologies. In the context of medical imaging, new and improved technologies including noninvasive imaging could be evaluated using instrumentation designed for humans.

The invention further provides methods of targeting the introduction of mutations into pig cells. These methods involve the steps of providing pig cells (e.g., fetal fibroblasts), using a recombinant adeno-associated viral (rAAV) vector (also referred to herein as an adeno-associated viral (AAV) vector) to deliver a gene targeting construct to the isolated pig cells, in the absence of cell detachment and reattachment, and selecting gene-targeted clones. The cells are in culture for 30 days or less (e.g., 20 days or less in the Examples) during the targeting construct delivery and selection steps. These methods can be used, for example, for the introduction of a mutation into an ATM gene in the pig cell. Information concerning other examples of mutations that can be used in the present invention, as well as the use of the present methods to inactivate or replace genes (e.g., to replace pig genes with human genes), is provided below.

By “donor cell” is meant a cell from which a nucleus or chromatin material is derived, for use in nuclear transfer. As is discussed elsewhere herein, nuclear transfer can involve transfer of a nucleus or chromatin only, as isolated from a donor cell, or transfer of an entire donor cell including such a nucleus or chromatin material.

By “genetic modification,” “mutation,” or “disruption” of a gene (e.g., an ATM gene) is meant one or more alterations in gene sequences (including coding sequences and non-coding sequences, such as introns, promoter sequences, and 5′ and 3′-untranslated sequences) that alter the expression or activity of this gene by, for example, insertion (of, e.g., heterologous sequences, such as selectable markers, and/or termination signals), deletion, frame shift mutation, silent mutation, nonsense mutation, missense mutation, point mutation, or combinations thereof. In one example, the amino acid sequence encoded by the nucleic acid sequence has at least one amino acid altered as compared to a naturally-occurring sequence. Examples of mutations include the insertion of a polynucleotide into a gene, the deletion of one or more nucleotides from a gene, and the introduction of one or more base substitutions into a gene. In one embodiment of the present invention, modifications of ATM gene sequences are those that lead to one or more features or symptoms of A-T in transgenic animals including a mutation in, or disruption of, one of the ATM alleles. In another embodiment of the present invention, modifications of ATM gene sequences are those that lead to one or more features or symptoms of A-T in transgenic animals including a mutation in, or disruption of, both ATM alleles. As is discussed elsewhere herein, the modifications in the two ATM alleles of such animals can be identical or different. Further, the modifications can result in a complete lack of functional ATM production, or can result in diminished functional ATM production, as may be characteristic of a range of A-T phenotypic severity.

Examples of such mutations include any such mutations known in the art, for example, those listed at http://chromium.liacs.nl/LOVD2/home.php?select_db=ATM.

In one example, a mutation is introduced by the insertion of a polynucleotide (for example, a positive selection marker, such as an antibiotic resistance gene (e.g., a neomycin resistance gene)) into an endogenous gene. Optionally, a mutation that is introduced into such an endogenous gene reduces the expression of the gene. If desired, the polynucleotide may also contain recombinase sites flanking the positive selection marker, such as loxP sites, so that the positive selection marker may be removed by a recombinase (e.g., cre recombinase).

By “homologous” genes is meant a pair of genes from two animal species that encode proteins having similar functional and physical properties. The proteins encoded by homologous genes are often very similar in structure and function (although not always), and typically have a common evolutionary origin. In one embodiment, the sequence identity is typically equal to or greater than 80%, equal to or greater than 90%, equal to or greater than 95%, or equal to or greater than 98% between two gene homologs. One example of a homologous gene pair is the porcine ATM and human ATM gene locus.

By “orthologous” genes or “orthologs” is meant genes that are separated by a speciation event wherein one ortholog may be substituted by genetic engineering into its corresponding gene in another species.

By animal “knock-out” is meant an animal (for example, a pig or mouse; also see other animals described herein) having a genome in which the function of a gene has been disrupted, or “knocked-out.” A common method of producing disabled genes using recombinant DNA technology involves inserting an antibiotic resistance gene into the normal DNA sequence of a clone of the gene of interest by homologous recombination. This disrupts the action of the gene, thereby preventing it from leading to the production of an active protein product. A cell (or cell nucleus) in which this transfer is successful can be injected into a recipient cell (e.g., an enucleated oocyte) to generate a transgenic animal by nuclear transfer. In another approach, the cell is injected into an animal embryo, producing a chimeric animal. These animals are bred to yield a strain in which all of the cells contain the knocked-out gene.

By “heterozygous knock-out non-human mammal” is meant a mammal other than a human in which one of the two alleles of an endogenous gene (such as the ATM gene) have been genetically targeted, or knocked out, resulting in a marked reduction or elimination of expression of a functional gene product, which is achieved by gene deletion or disruption.

By “homozygous knock-out non-human mammal” is meant a mammal other than a human in which the two alleles of an endogenous gene (such as the ATM gene) have been genetically targeted, or knocked out, resulting in a marked reduction or elimination of expression of a functional gene product, which is achieved by gene deletion or disruption. According to the invention, the genetic targeting event at both alleles may or may not be the same. Thus, a non-human animal, in which the two alleles of an endogenous gene (such as an ATM gene) have been genetically targeted by two different targeting vectors resulting in the null expression of the gene, would be considered as being a homozygous knock-out non-human mammal.

An example of a “knock-in mutation” is one resulting in the insertion of a mutation into an endogenous gene.

By “recipient cell” is meant a cell into which a donor cell, a donor cell nucleus, or donor cell chromatin is introduced. In one preferred embodiment, recipient cells are enucleated prior to nuclear transfer. Examples of recipient cells include oocytes, fertilized zygotes, and two-cell embryos.

By “transgenic, large non-human animal” is meant any non-human animal that includes a genetic modification, as defined herein. Examples of such animals include animals other than mice such as, for example, ungulates. Examples of ungulates that can be used in the invention include members of the orders Perissodactyla and Artiodactyla, such as any members of the family Suidae, and in particular any member of the genus Sus, such as Sus scrofa, which is also known as the domestic pig or a subspecies thereof (Sus scrofa domestica). Examples of Sus scrofa domestica breeds that can be used in the present invention include Landrace, Hampshire, Duroc, Chinese Meishan, Berkshire, Pietrain and Yorkshire. Examples of miniature pigs that can be used in the present invention include Ossabaw, Hanford, Sinclair, Libechov, Goettingen, Yucatan, Bama Xiang Zhu, Wuzhishan, and Xi Shuang Banna. In addition to porcines, additional ungulates that can be used in the invention include bovines, ovines, and caprines. Thus, for example, the invention can include the use of cows (e.g., Bos taurus or Bos indicus), sheep, goats, buffalos, antelopes, oxen, horses, donkeys, mule, deer, elk, caribou, water buffalo, camels, llama, alpaca, and elephants.

The invention provides several advantages over the state of the art, as it provides large, non-human animal models that can be used in the identification and characterization of therapies for genetic diseases, for example, the present invention describes the development of the first gene-targeted large animal model of a human neuromuscular disease. One example of such a disease is A-T, which, as discussed above, is a devastating disease, leading to cerebellar ataxia, dilation of small blood vessels in the eyes and skin and a predisposition to cancer, for example.

Mouse models of A-T have provided an insight into some consequences of ATM dysfunction, displaying a variety of growth defects, immunological abnormalities, sensitivity to ionizing radiation, and a predisposition to cancer, but are of limited use for the study of neurological disease since the mice do not exhibit the same severe neurological defects that are characteristic of A-T disease. See, e.g., Barlow, C., et al., Cell, 1996. 86(1): p. 159-71; Elson, A., et al., Proc Natl Acad Sci USA, 1996. 93(23): p. 13084-9; Xu, Y., et al., Genes Dev, 1996. 10(19): p. 2411-22; Herzog, K. H., et al., Science, 1998. 280(5366): p. 1089-91; Borghesani, P. R., et al., Proc Natl Acad Sci USA, 2000. 97(7): p. 3336-41. Atm-deficient mice fail to demonstrate an ataxic phenotype, and often die of other complications of ATM dysfunction, such as lymphoma, rather than from neurological disease. A large animal model, such as the pig, holds the promise of a more accurate disease model given the similarities that pigs and humans share in terms of development, anatomy, and physiology, and in particular, similarities in brain development such as perinatal growth spurt and brain structure (see, e.g., Pond, W. G., et al., Proc Soc Exp Biol Med, 2000. 223(1): p. 102-8; Jelsing, J., et al., J Exp Biol, 2006. 209(Pt 8): p. 1454-62). An improved model of A-T would increase our understanding of A-T disease mechanisms and provide a more relevant setting in which to test new therapeutic interventions.

A porcine model offers several advantages over murine models. First, gene targeting provides an opportunity to introduce almost any desired mutation. This will allow for the targeting of “hotspot” regions that represent a broader range of patient-specific mutations. Second, somatic cell nuclear transfer (SCNT), sometimes referred to as cloning, offers the unique ability to produce genetically identical A-T pigs, as well as genetically identical control animals (with the exception of the specific mutation of interest). This will reduce phenotypic variability and allow researchers to study specific mechanisms and treatments without concern of extraneous genetic factors. Conversely, the wide range of pig breeds also allows for genetic outcrossing as a means to identify and study modifier genes.

Availability of ATM-targeted animal models, for example, pig models, will allow investigators to address key problems that have persisted unresolved for years. As a result, it will be possible to develop new treatments, medical devices, therapies, and preventions for A-T. Further, given the close physiological relationship between humans and large animals, such as pigs, there is an increased likelihood that results obtained using the animal models of the invention can be applied to humans, relative to other animal models. For example, as stated above, mouse models of A-T fail to demonstrate an ataxic phenotype, and often die of other complications of ATM dysfunction, such as lymphoma, rather than from neurological disease. This is likely due to genetic, biochemical, and physiological differences between mice and humans. Specifically with respect to pigs, it is noted that pigs and humans have anatomical, histological, biochemical, and physiologic similarities.

The invention thus can be used to provide substantial benefits in the treatment of diseases and conditions caused by or associated with gene mutations, such as A-T. Other features and advantages of the invention will be apparent from the drawings, the detailed description, the experimental examples, and the claims.

### BRIEF DESCRIPTION OF THE DRAWINGS

This patent application file contains at least one drawing executed in color. Copies of this patent application publication with color drawing(s) will be provided by the Office upon request and payment of the necessary fee.

FIG. 1 is a schematic drawing showing one example of a method for generating ATM-targeted pigs. Fibroblasts are obtained from day 35 Yucatan fetuses. The targeting vector (with, for example, an ATM mutation indicated by the asterisk) is introduced to fetal fibroblasts via AAV infection. In one example, the mutation is the disruption of exon 57 of ATM. Properly targeted cells are identified by PCR and Southern blot. Following nuclear transfer and fusion and/or activation, nuclear transfer embryos are transferred to recipient animals. After a 114 day gestation period, the resulting piglets have one ATM-targeted allele.

FIG. 2 shows a schematic drawing (not drawn to scale) of the gene targeting vector (SEQ ID NO: 1) used to disrupt porcine ATM. Exons 55-58 of porcine ATM are depicted in black boxes. Neo^{R} contains a neomycin resistance cDNA (NEO) driven by the phosphoglycerate kinase (PGI) promoter and flanked by loxP sites (LOXP). The rAAV inverted terminal repeats (ITRs) are also shown as diagonal striped boxes. Each homology arm is about 1.4 kb in length.

FIG. 3 shows PCR screen identified ATM-targeted cells. FIG. 3A shows a representative 96-well gel containing a PCR-positive clone (boxed). The other wells represent Neo^{R} clones resulting from random integration, or in the case of lighter bands, leftover DNA from dead cells. Each PCR-positive clone was re-electrophoresed on a conventional agarose gel to confirm proper size, as shown, for example, in FIG. 3B. Expected sizes were 1.7 kb for targeted ATM and 3.8 kb for an internal control sequence (wild-type LDLR). Lanes 1-7 represent ATM-targeted cells (lane 3 represents a mixed population with very few ATM-targeted cells; targeted band is very faint). FIG. 3C shows a sequence (SEQ ID NO: 2) chromatogram of the site of ATM disruption by the Neo^{R} cassette. The engineered termination stop codon is noted.

FIG. 4 shows heterozygous (ATM+/−) piglets. FIG. 4A shows piglets from the first ATM-targeted litter. FIG. 4B shows a Genomic Southern blot of DNA from ATM-targeted piglets. On the left photo, XmnI digested genomic DNA was hybridized with a probe that detects porcine ATM downstream of the targeting vector boundary. The ATM-targeted allele produced an approximately 7.2 kb band, and the wild-type band is approximately 5.5 kb. In the right photo, the same DNA was hybridized with a probe that detects the Neo^{R} cassette, yielding only the targeted 7.2 kb band in properly targeted piglets. Lanes 1-5 contain DNA from individual cloned piglets (Piglets 1, 2, and 4 are ATM+/−). Lane 6 contains XmnI-digested DNA from a wild-type pig.

FIG. 5 shows representative genomic Southern blot from three ATM genotypes (ATM+/+, ATM+/−, and ATM−/−). In the left photo, XmnI digested genomic DNA was hybridized with a probe that detects porcine ATM downstream of the targeting vector boundary. The ATM-targeted allele produced an approximately 7.2 kb band, and the wild-type band is approximately 5.5 kb. In the right photo, the same DNA was hybridized with a probe that detects the Neo^{R} cassette, yielding only the targeted 7.2 kb band in properly targeted piglets.

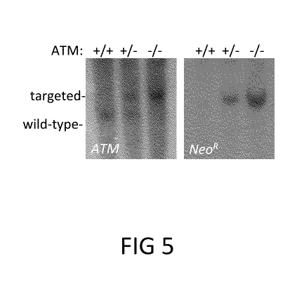

FIG. 6 shows a representative western blot of ATM. ATM is ˜370 kDa and β-tubulin is 51 kDa. Lanes 1-3 contain protein from ATM+/+ pigs, while lanes 4-6 contain protein from ATM−/− pigs. This confirms that ATM−/− pigs do not produce normal ATM protein.

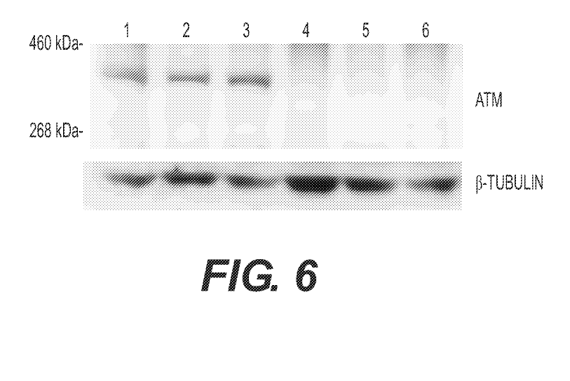

### DETAILED DESCRIPTION OF THE INVENTION

The invention provides animal models of human disease (e.g., A-T), which can be used in the identification and characterization of approaches for treating the diseases and conditions. As is discussed further below, the animal models of the invention are large, non-human animals, such as pigs, which have been genetically modified to include one or more mutations in a gene associated with a particular disease or condition, for example, the Ataxia-Telangiectasia Mutated gene. The genetic modifications can result in the animals having one or more symptoms characteristic of the disease or condition. Animals exhibiting such symptoms are particularly advantageous in the development of therapeutic approaches, as candidate drugs and other approaches to treatment can be evaluated for effects on the symptoms in such animals. Thus, in addition to the animal models themselves, the invention also provides methods of using the animals for identifying and characterizing treatments.

Further, the invention includes methods of making transgenic, large non-human animal models and cells that can be used in these methods. The animal models systems, methods, and cells of the invention are described further, below.

In one embodiment, the invention provides a heterozygous or homozygous knock-out non-human mammal (e.g., a pig). In one example, the invention provides a pig with its endogenous porcine ATM gene knocked-out (i.e., an ATM+/− or ATM−/− pig.)

In addition to animals including knock-outs or mutations in endogenous genes, the invention also includes transgenic, large non-human animal models of human diseases and conditions (e.g., pigs), in which one or more endogenous genes associated with the diseases or conditions are knocked-out (i.e., genetically altered in such way as to inhibit the production or function of the products of these genes) and replaced with a comparable wild-type or mutated gene derived from a different animal (e.g., a human). In one example, a pig with its endogenous porcine ATM gene knocked-out, expresses a mutant human ATM transgene. Alternatively, the human transgene may encode a normal, wild-type copy of a gene of interest (e.g., ATM). These embodiments of the invention are especially useful for the generation of non-human animal models of human diseases and conditions that can be used to test existing and potential therapeutics that may only (or may preferentially) modulate or treat the disease when contacting, or being in the presence of, human copies of the disease gene or protein in question.

The invention is described herein in reference to animal models of Ataxia-Telangiectasia, which are generated by mutation, deletion or replacement of the ATM gene. However, the methods of the invention are also applicable to the development of animal models of additional diseases and conditions.

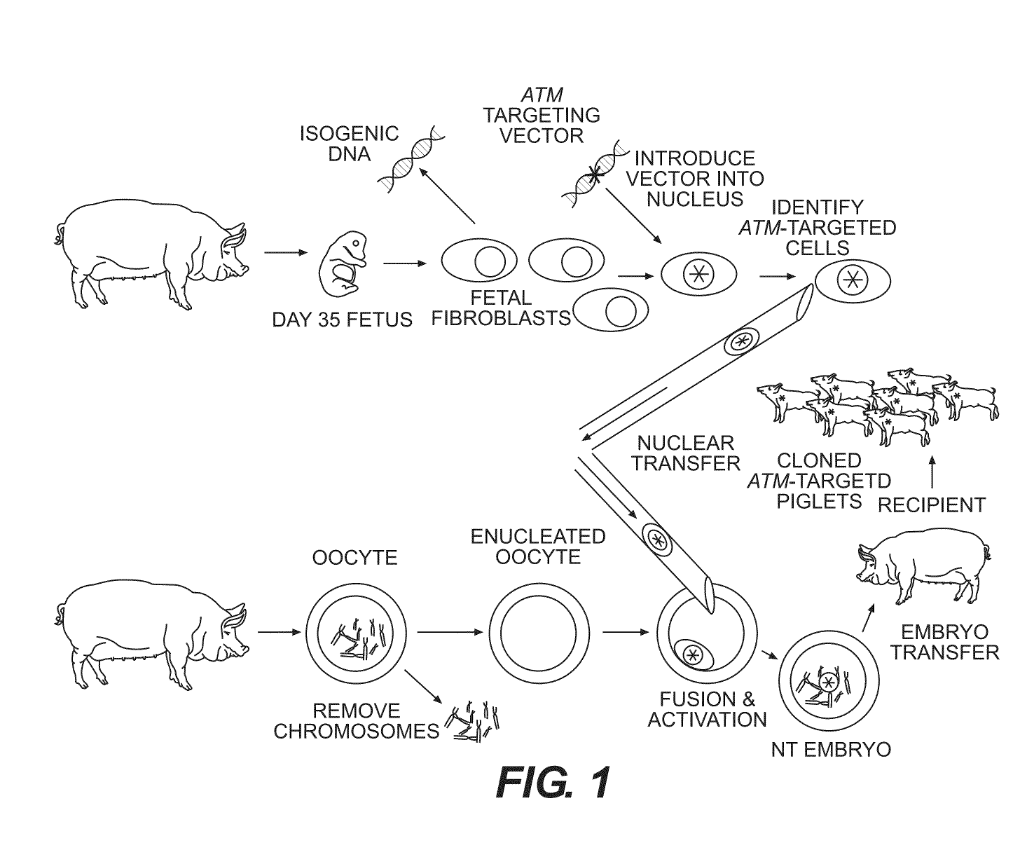

The transgenic animals of the invention can be made using the following general strategy, which combines gene targeting and somatic cell nuclear transfer (SCNT), also known as cloning. Briefly, the genome of a cell (e.g., a fetal fibroblast) from an animal of interest, such as a pig, is genetically modified by, for example, gene targeting by homologous recombination, to create a “donor cell.” According to the methods of the invention, the genetic modification results in at least partial inactivation of a gene associated with a particular disease or condition (e.g., an ATM gene in Ataxia-Telangiectasia), as will be described in further detail below. The nucleus of such a genetically modified donor cell (or the entire donor cell, including the nucleus) is then transferred into a so-called “recipient cell,” such as an enucleated oocyte. After activation and, typically, a brief period of in vitro culture, the resulting embryo is implanted into a surrogate female in which development of the embryo proceeds. This approach is illustrated with respect to pigs in FIG. 1. Typically, the donor cell, oocyte, and surrogate female are of the same species, but the sources can be different species, as is known in the art.

Similar procedures have been used to develop two different gene-targeted porcine models of cystic fibrosis, and a model of atherosclerosis. See, e.g., Rogers, C. S., et al. J Clin Invest 2008, 118 (4), 1571-7; Rogers, C. S., et al. Science 2008, 321 (5897), 1837-41; U.S. Pat. No. 7,989,675; U.S. patent application Ser. Nos. 13/288,720, 13/368,312 and 13/624,967. However, these methodologies are still at the forefront of the large animal transgenic field, and this is the first application of such technologies for a pediatric neurological disease. Furthermore, the use of animal cloning technologies has yet to be fully realized for biomedical research. In studies for which genetic variability can be problematic, the ability to generate genetically identical A-T pigs, as well as control animals that are genetically identical except for the specific mutation, provides a resource that allows researchers to study specific mechanisms and treatments without concern of extraneous genetic factors.

Large animal models for A-T (for example, the ATM-targeted pig), will also provide a basis for research into the neurological aspects of A-T, which is currently lacking in mouse models.

Details of methods for making large genetically modified animals according to the invention are provided below. Additional information concerning methods for making genetically modified pigs and other large animals is known in the art and can also be used in the present invention (see, e.g., U.S. Pat. No. 7,547,816; and WO 2005/104835; Prather et al., Reproductive Biology and Endocrinology 1:82, 1-6, 2003; Hao et al., Transgenic Res. 15:739-750, 2006; Li et al., Biology of Reproduction 75:226-230, 2006; Lai et al., Nature Biotechnology 24(4):435-436, 2006; Lai et al., Methods in Molecular Biology 254(2):149-163, 2004; Lai et al., Cloning and Stem Cells 5(4):233-241, 2003; Park et al., Animal Biotechnology 12(2):173-181, 2001; Lai et al., Science 295:1089-1092, 2002; Park et al., Biology of Reproduction 65:1681-1685, 2001).

The transgenic animals of the invention can be any transgenic, large non-human animal. In one embodiment, the transgenic animal is a pig. Pigs share many similarities with humans including anatomy, biochemistry, physiology, size (particularly miniature pig breeds), lifespan, and genetics. The pig has proven to be an excellent model for obesity, diabetes, alcoholism, hypertension, skin physiology, intestinal function, nutrition, and injury (see, e.g., Rogers, C. S., et al. Am J Physiol Lung Cell Mol Physiol 2008, 295 (2), L240-63). Recently, two porcine models of cystic fibrosis were developed that demonstrate all of the clinical manifestations of the human disease, including meconium ileus, pancreatic insufficiency, and lung disease. See, e.g., Rogers, C. S., et al. Science 2008, 321 (5897), 1837-41; Meyerholz, D. K., et al. Am J Respir Crit Care Med 2010, 182 (10), 1251-61; Stoltz, D. A., et al. Sci Transl Med 2010, 2 (29), 29ra31; and Meyerholz, D. K., et al. Am J Pathol 2010, 176 (3), 1377-89. In addition, similarity of porcine and human organs has led to a large effort to develop them as a source of organs for xenotransplantation (see, e.g., Cooper, D. K., et al. Annu Rev Med 2002, 53, 133-47). Furthermore, the reproductive characteristics of swine are favorable for their use as a model (See Table 1). Their relatively fast maturation rate and the large number of offspring generated from a single sow in one year allow a colony to rapidly expand. Finally, for an A-T model, pigs share similarities in brain development and like humans, are described as having a primarily perinatal developmental period, and brain structures that much more similar to that of humans than mice.

TABLE 1

| | | | Offspring | | |
|---|---|---|---|---|---|
| | Gestation | Sexual | per | Deliveries | Offspring |
| Species | time | maturity | delivery | per year | per year |
| Mouse | 20-22 d | 40-60 d | ~6 | ~17 | 100 |
| NH primate | 150-175 d | 4-5 yr | 1 | 2 | 1-2 |
| Minipig | 114 d | 5-6 mo | 4-7 | ~3 | 12-21 |

TABLE 1. Reproductive characteristics of several species (values are approximate).

Additionally, porcine ATM shows remarkable similarity to human in gene sequence and genomic structure. The full-length porcine ATM protein has been characterized and is 88% identical to human ATM. See, e.g., Rogatcheva M. B., et al., Gene. 2007 Dec. 15; 405(1-2):27-35. Epub 2007 Aug. 30.

The invention includes animals in which only one allele of a targeted gene (e.g., ATM) is disrupted, with the other allele remaining unaffected. These animals, which are referred to herein as “heterozygous” or “hemizygous” animals, can be used, for example, as models to study the development or progression of a disease (for example, Ataxia-Telangiectasia or cancer) in heterozygous animals. Further, these animals can be used in breeding approaches to generate homozygous mutants, if desired, for example, in the case of diseases caused by homozygous recessive mutations. Additionally, these animals can be used in breeding approaches to generate, for example, a cancer model when crossed with TP53-mutated pigs, such as those described in co-pending U.S. Appln. Nos. 61/788,518, filed on Mar. 15, 2013, and 61/951,896, filed on Mar. 12, 2014, each of which is hereby incorporated by reference in its entirety.

The heterozygous animals of the present invention can also be used as animal models themselves, for example, in the case of diseases caused by autosomal dominant mutations, or where disruption of one allele of the targeted gene may result in some phenotypic expression of the mutation that is less severe than disruption of both alleles. For example, the heterozygous pigs of the present invention can be used to study cancer susceptibility in animals with one disrupted allele as compared to ATM−/− pigs. Humans with ATM mutations or polymorphisms in one allele have been shown to be more prone to lung, breast, and pancreatic cancer than those without ATM mutations or polymorphisms. See, e.g., Shen L., et al., Mol Biol Rep. 2012 May; 39(5):5719-25. doi: 10.1007/s11033-011-1381-2. Epub 2011 Dec. 28.

Also included in the invention are homozygous mutant animals, in which both alleles of a target gene (e.g., ATM) are disrupted or mutated, by the same or different mutations. In addition to being obtainable by breeding approaches involving hemizygous animals, homozygous mutant animals can also be obtained using an approach in which a cell (e.g., a fetal fibroblast) including a mutation in one allele, such as a cell obtained from an animal produced using the method summarized above, is subjected to gene targeting by homologous recombination to achieve modification of the remaining allele. The resulting donor cell can then be used as a source of a modified nucleus for nuclear transfer into a recipient cell, such as an enucleated oocyte, leading to the formation of a homozygous mutant embryo which, when implanted into a surrogate female, develops into a homozygous mutant animal.

A target gene (e.g., an ATM gene) can be subject to genetic modification in any appropriate cell type of a species for which it is desired to create an animal model of a disease associated with mutation of the gene, according to the invention. As is understood in the art, it is necessary to be able to culture and carry out homologous recombination in a cell that is to be used as a donor cell. A particular example of such a cell, which is described in more detail below in connection with pigs, in the experimental examples, is the fetal fibroblast. These cells can be obtained using, for example, the approach described in U.S. Pat. No. 7,547,816 and other references cited herein.

The invention also includes the use of other cell types that may be present in the cell preparations obtained using the method described in U.S. Pat. No. 7,547,816. Additional examples of cells that can be used as donor cells in making the transgenic animals of the invention include other fetal cells, placental cells, or adult cells. Specific examples of such cells for gene targeting include differentiated cells such as fibroblasts, epithelial cells, neural cells, epidermal cells, keratinocytes, hematopoietic cells, melanocytes, chondrocytes, B-lymphocytes, T-lymphocytes, erythrocytes, macrophages, monocytes, placental, and muscle cells.

If a cell to be genetically altered is derived from an embryo or a fetus, the cell (e.g., a fetal cell or placental cell) can be isolated at any time during the gestation period until the birth of the animal, which may or may not be itself genetically altered. In the case of a pig, such cells can be obtained, for example, between 20 to 90 days of gestation, between 25 to 60 days of gestation, between 30 to 45 days of gestation, or between 35 to 40 (e.g., at 35 days) of gestation. The time periods for obtaining cells from other animals is known in the art (see, e.g., U.S. Pat. Nos. 7,420,099 and 7,928,285).

Gene targeting carried out to make the cells and animals of the invention can result in gene inactivation by disruption, removal, modification, or replacement of target gene sequences. For example, inactivation can take place by the insertion of a heterologous sequence and/or a stop codon into a target gene. A specific example of this type of inactivation, in the context of an ATM gene, is described in the experimental examples, below. As is known in the art, inserted sequences can replace previously existing sequences in a gene or can be added to such sequences, depending on the design of the targeting construct. In another example, deletion of a sequence using homologous recombination results in a frameshift mutation that yields a prematurely truncated and non-functional protein.

As is known in the art, the design of targeting constructs can be altered, depending upon whether it is desired to completely knock out the function of a gene or to maintain some level of reduced function. In the case of ATM, for example, complete knock out of function would be consistent with the most severe forms of A-T in which there is no ATM present. However, other less dramatic changes may be desirable for the generation of models of disease maintaining some ATM function. Such changes may be achieved by, for example, replacement with sequences that are identical to wild-type sequences, except for the presence of specific mutations giving rise to features of the target disease. In other approaches, coding sequences are not altered or are minimally altered and, rather, sequences impacting expression of a target gene, such as promoter sequences, are targeted. In any case, selectable marker insertion is often desirable to facilitate identification of cells in which targeting has occurred. If desired, such markers or other inserted sequences can later be removed by, e.g., cre-lox or similar systems.

A “humanized” ATM model (i.e., an ATM−/− animal expressing a mutant human ATM transgene) can be made numerous ways, including, but not limited to: i) introducing a mutant human ATM cDNA, partial mutant human ATM gene, or entire human ATM gene carrying a mutation into animal (e.g., porcine) ATM−/− cells, selecting for mutant human ATM gene insertion, and using these cells as nuclear donors in somatic cell nuclear transfer, and ii) introducing a mutant human ATM cDNA, partial mutant human ATM gene, or entire human ATM gene carrying a mutation to animal ATM−/− cells into matured oocytes, fertilizing, then transferring to a recipient female.

As is known in the art, targeted gene modification requires the use of nucleic acid molecule constructs having regions of homology with a targeted gene (or flanking regions), such that integration of the construct into the genome alters expression of the gene, either by changing the sequence of the gene and/or the levels of expression of the gene. Thus, to alter a gene, a targeting construct is generally designed to contain three main regions: (i) a first region that is homologous to the locus to be targeted (e.g., the ATM gene or a flanking sequence), (ii) a second region that is a heterologous polynucleotide sequence (e.g., encoding a selectable marker, such as an antibiotic resistance protein) that is to specifically replace a portion of the targeted locus or is inserted into the targeted locus, and (iii) a third region that, like the first region, is homologous to the targeted locus, but typically is not contiguous with the first region of the genome. Homologous recombination between the targeting construct and the targeted wild-type locus results in deletion of any locus sequences between the two regions of homology represented in the targeting vector and replacement of that sequence with, or insertion into that sequence of, a heterologous sequence that, for example, encodes a selectable marker. Use of such promoters may not be required in cases in which transcriptionally active genes are targeted, if the design of the construct results in the marker being transcribed as directed by an endogenous promoter. Exemplary constructs and vectors for carrying out such targeted modification are described herein. However, other vectors that can be used in such approaches are known in the art and can readily be adapted for use in the invention.

In order to facilitate homologous recombination, the first and third regions of the targeting vectors (see above) include sequences that exhibit substantial identity to the genes to be targeted (or flanking regions). By “substantially identical” is meant having a sequence that is at least 80%, preferably at least 85%, preferably at least 90%, more preferably at least 95%, even more preferably at least 98%, and even more preferably 100% identical to that of another sequence. Sequence identity is typically measured using BLAST® (Basic Local Alignment Search Tool) or BLAST® 2 with the default parameters specified therein (see, Altschul et al., J. Mol. Biol. 215: 403-410, 1990; Tatiana et al., FEMS Microbiol. Lett. 174: 247-250, 1999). These software programs match similar sequences by assigning degrees of homology to various substitutions, deletions, and other modifications. Thus, sequences having at least 80%, preferably at least 85%, preferably at least 90%, more preferably at least 95%, even more preferably at least 98%, and even more preferably 100% sequence identity with the targeted gene loci can be used in the invention to facilitate homologous recombination.

The total size of the two regions of homology (i.e., the first and third regions noted above) can be, for example, approximately between 2-25 kilobases (for example, approximately between 4-20 kilobases, approximately between 5-15 kilobases, or approximately between 6-10 kilobases), and the size of the second region that replaces a portion of the targeted locus can be, for example, approximately between 0.5-5 kilobases (for example, approximately between 1-4 kilobases or approximately between 3-4 kilobases).

The targeting constructs can be included within any appropriate vectors, such as plasmid or viral vectors (e.g., adenovirus or adeno-associated virus (AAV) vectors), which can be introduced into cells using standard methods including, for example, viral transduction, electroporation, or microinjection. One preferred example of the invention, which is described in detail in the experimental examples, below, employs a recombinant adeno-associated viral vector (rAAV), which can be made by standard methods or produced commercially.

Recombinant adeno-associated virus has been used to deliver gene targeting vectors to cell lines and primary cells (see, e.g., Russell, D. W., et al. Nat Genet 1998, 18 (4), 325-30). For example, rAAV has been used to introduce two different targeted modifications to the porcine CFTR gene. See, e.g., Rogers, C. S., et al. J Clin Invest 2008, 118 (4), 1571-7, U.S. Pat. No. 7,989,675 and U.S. patent application Ser. No. 12/283,980.

The use of a rAAV to deliver the targeting construct offers many benefits. First, rAAV1 (and other rAAV serotypes) infects pig fetal fibroblasts with nearly 100% efficiency. See, e.g., Rogers, C. S., et al. J Clin Invest 2008, 118 (4), 1571-7. Second, rAAV infection of pig fetal fibroblasts results in little or no cell toxicity. Third, rAAV infection results in the delivery of a single-stranded gene-targeting construct directly to the nucleus, the amount of DNA per cell is small, and it can infect many cell types. Importantly, the ratio of homologous recombination events to random integrations is more favorable than that seen with electroporation of lipid-mediated transfection. See, e.g., Vasquez, K. M., et al. Proc Natl Acad Sci USA 2001, 98 (15), 8403-10.

The methods of the invention, employing rAAV vectors, resulted in high levels of gene targeting efficiency in these somatic cells, as compared to prior methods. Central to the methods of the invention is the fact that the entire procedure was performed in a time-sensitive manner, because excessive cell culture time (for example, more than 30 days) negatively impacts nuclear transfer efficiency (Lai et al., Cloning and Stem Cells 5(4):233-241, 2003). Following fibroblast harvest from day 35 fetuses, the cells were frozen within 48 hours. The use of an AAV vector to deliver the gene targeting construct allowed targeting to begin 24 hours after thawing cells and required no cell detachment and re-attachment, which is required in other methods. Multiple cell detachment and re-attachment events (trypsinization) are thought to decrease the ability of a cell to serve as a nuclear donor in nuclear transfer. Further, G418 selection in 48 96-well plates prevents the need for the more conventional, time-consuming isolation of resistant clones with cloning rings. The screen for gene targeted clones was designed such that all positive clones could be identified and frozen within a 3-5 day period. All clones were frozen by day 18, therefore the cells have been in culture approximately 20 days since being harvested from the fetus. In this aspect of the invention, reduction of the time in culture increases the likelihood that cells used as nuclear donors are viable, normal, and euploid.

Accordingly, the invention provides a method of gene-targeting cells, such as pig cells (e.g. pig fetal fibroblasts), in which the number of days in culture (during which targeting and selection takes place) is preferably less than 30 days, preferably 25-29 days, preferably 20-24 days, and more preferably 19, 18, 17, 16, 15, or fewer days. To facilitate this method, the selection can take place in multi-well plates, as described further below. Further, the cells may be frozen shortly after harvest (for example, within 24, 48 or 96 hours). After cell thawing (or after harvest, if the cells are not previously frozen), gene targeting with an rAAV vector can be carried out within, for example, 12, 24, 36 or 48 hours, without the use of multiple detachment/re-attachment events, and selection can proceed in an expedited manner, such as by use of multi-well plates (e.g., 96 well plates), prior to freezing.

Other types of vectors, or more specifically other types of targeting construct delivery methods, are also available to those of skill in the art and may be used in the present invention. Such methods include cell transfection methods, including calcium phosphate, lipofection, electroporation, and nuclear injection, all of which can be used to deliver the targeting construct. If the gene is transcriptionally active in the cell being used, then a promoterless selectable strategy can be employed, so that antibiotic resistance will only be found in cells that have had a recombination event within the transcribed unit.

Genetically targeted cells are typically identified using a selectable marker, such as neomycin. If a cell already contains a selectable marker, however, a new targeting construct containing a different selectable marker can be used. Alternatively, if the same selectable marker is employed, cells can be selected in the second targeting round by raising the drug concentration (for example, by doubling the drug concentration), as is known in the art. As is noted above, targeting constructs can include selectable markers flanked by sites facilitating excision of the marker sequences. In one example, constructs can include loxP sites to facilitate the efficient deletion of the marker using the cre/lox system. Use of such systems is well known in the art, and a specific example of use of this system is provided below, in the experimental examples.

Upon obtaining cells in which a target gene (e.g., an ATM gene) has been targeted (one or both alleles, as described above), nuclear transfer can be carried out. Optionally, the genetically modified nuclear donor cells can be frozen prior to nuclear transfer. Recipient cells that can be used in the invention are typically oocytes, fertilized zygotes, or two-cell embryos, all of which may or may not have been enucleated. Typically, the donor and the recipient cells are derived from the same species. However, it is possible to obtain development from embryos reconstructed using donor and recipient cells from different species.

Recipient oocytes can be obtained using methods that are known in the art or can be purchased from commercial sources. As is known in the art, the donor nucleus or the donor cell itself can be injected into the recipient cell or injected into the perivitelline space, adjacent to the oocyte membrane. The nuclear transfer complex formed in this manner can be activated by standard methods, which may involve electrical fusion/activation or electrical fusion/chemical activation, as is described further below. Further processing of the nuclear transfer complex, including implementation of the complexes into surrogate mothers, is described further below.

The transgenic animals of the invention can be used in the identification and characterization of drug and other treatment methods for the disease or condition associated with mutation of the gene targeted according to the invention. In these methods, for example, a candidate therapeutic agent can be administered to an animal and the impact of the agent on a feature of the disease exhibited by the animal can be monitored. Optionally, the methods can also involve exposure of the animals to environmental or other conditions known to contribute to or exacerbate the disease or condition. For example, in the case of Ataxia-Telangiectasia, animal models having impaired function in the ATM gene can be used to monitor the effect of a therapeutic agent, such as a drug, on ATM function or production. In another example, gene- and cell-based therapies for Ataxia-Telangiectasia can be administered in such an animal and the animal may be monitored for the effects on production and function of ATM, and further can be used to assess the effect and the impact on progression (or reversal) of Ataxia-Telangiectasia.

With the porcine model of the invention, it is possible to test hypotheses that lead to new treatments, diagnostics, protocols, imaging technologies and medical devices, and to evaluate potential therapies for Ataxia-Telangiectasia. Likely activities involving the present invention may include evaluating current and future therapeutics for toxicity, pharmacokinetics and efficacy within the same animal. Medical devices makers may study efficacy of products in a relevant, diseased setting. And in the context of medical instruments, noninvasive imaging may be evaluated to diagnose and chart the progression of Ataxia-Telangiectasia.

Availability of animal models for Ataxia-Telangiectasia allows new investigations and tests of therapeutics in the immune system and organs affected primarily or secondarily by Ataxia-Telangiectasia, including, but not limited to the cerebellum, eyes, and lungs. The screening methods of the invention can be carried out to test the efficacy of new compounds, combinations of new and old compounds, diagnostics, non-pharmaceutical treatments (such as gene- and cell-based therapies), medical devices, and combinations of the foregoing.

The following Examples are meant to illustrate the invention and are not meant to limit the scope of the invention in any way.

### EXPERIMENTAL EXAMPLES

### Example 1

### Yucatan Miniature Pigs and Cells for Gene Targeting

The Yucatan miniature pig was selected for development of an ATM model. While it possesses the same biological characteristics as domestic pigs, the Yucatan miniature pig is significantly smaller. Most domestic pig breeds reach 100 kg in less than six months and can achieve weights of 250-300 kg within a few years. Yucatan miniature pigs reach a full-grown size of 65-90 kg at two years of life, which is more similar to an adult human. Therefore, the Yucatan miniature pigs are less expensive to house and feed. Additionally, this breed is more docile in nature and better suited for interactions with researchers. See, e.g., Panepinto, L. M., et al., Lab Anim Sci 1986, 36 (4), 344-7.

Due to the lack of suitable porcine embryonic stem cell lines, the standard methods for producing gene-targeted mice are not applicable in pigs (Piedrahita, J. A., Theriogenology 2000, 53 (1), 105-16). Instead, gene targeting must be achieved in a somatic cell that is then used as a nuclear donor for SCNT. While numerous cell types can be used as nuclear donors, only fetal fibroblasts have been used to successfully create gene-targeted pigs. Fibroblasts previously obtained from male and female Yucatan miniature pig fetuses at day 35 of gestation were selected. Fibroblasts from the Yucatan breed behave similar to domestic pig fibroblasts in culture, gene transfer, and for SCNT (Estrada, J. L., et al. Cloning Stem Cells 2008, 10 (2), 287-96).

### Example 2

### Creation of an ATM Targeting Construct

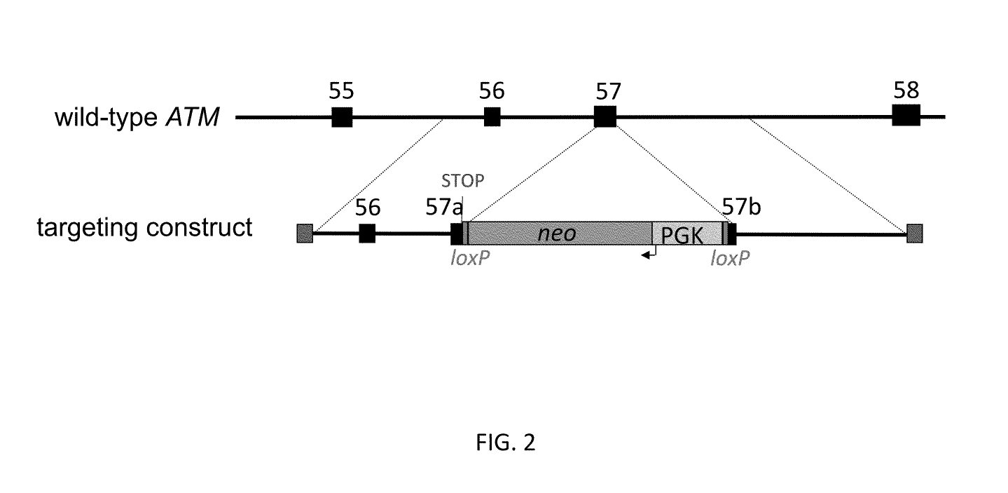

As mentioned above, porcine ATM has been sequenced and annotated, and the genomic structure is similar to the human gene. Homologous recombination was used to disrupt the endogenous ATM gene. Specifically, a neomycin-resistance cassette (Neo^{R}) was inserted into exon 57 of porcine ATM (FIG. 2). Exon 57 encodes a significant portion of the ATP-binding region within the kinase domain and it is known that a similar strategy to target ATM exons 57 and 58 in mice abolished ATM function (Herzog, K. H., et al., Science, 1998. 280(5366): p. 1089-91). A premature termination codon was also engineered immediately upstream of the Neo^{R} insertion. This strategy was adopted to maximize the likelihood of a non-functional ATM.

A plasmid carrying the ATM targeting vector was generated using standard molecular biology techniques. Proper sequence was confirmed by DNA sequence analysis. The plasmid was then submitted to the University of Iowa Gene Transfer Vector Core for production of recombinant adeno-associated virus (rAAV). rAAV was chosen because it has been used to efficiently deliver gene targeting vectors to cell lines and primary cells (Meyerholz, D. K., et al., Am J Respir Crit Care Med 2010, 182 (10), 1251-61). Additionally, rAAV has previously been used to engineer specific mutations in porcine CFTR and LDLR. See, e.g., Rogers, C. S., et al., J Clin Invest 2008, 118 (4), 1571-7; Rogers, C. S., et al., Science 2008, 321 (5897), 1837-41, U.S. Pat. No. 7,989,675; U.S. patent application Ser. Nos. 13/288,720, 13/368,312 and 13/624,967.

### Example 3

### Targeting ATM in Porcine Fetal Fibroblasts

Approximately 1.5×10⁶ Yucatan miniature pig fetal fibroblasts—both male and female—were infected with rAAV1 (MOI≅100,000) carrying the ATM targeting vector. After 24 hours, cells were transferred to a series of 96-well plates and G418 (100 μg/ml) was added to the media for selection of targeted cells. Fourteen days later, surviving cells were observed in 20-40% of wells, and each well of the 96-well plates were “replicated” by splitting among three plates: 1) 96-well culture plates for cell expansion, 2) 96-well culture plates for potential cryopreservation, and 3) 96-well PCR plates for cell lysis.

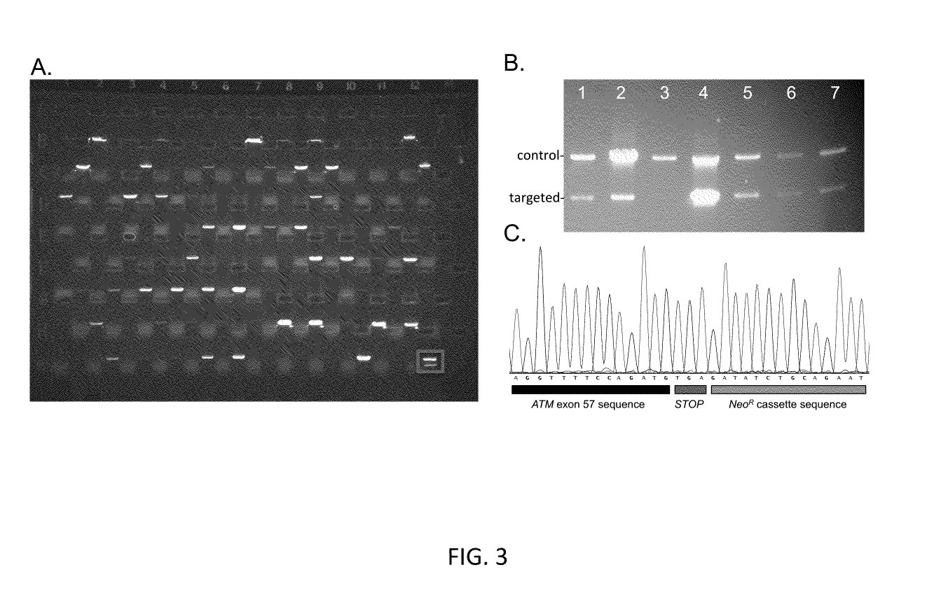

Cell lysates were screened by PCR to identify wells containing gene-targeted clones and any PCR-positive clones were frozen. (See FIGS. 3A and 3B). The PCR reaction contained two primer pairs—one pair amplified only the properly targeted ATM allele, the other amplified a portion of the porcine LDLR gene as an internal PCR control. PCR identified 17 ATM+/− male cell lines and 11 ATM+/− female cell lines.

By the time ATM-targeted cells were frozen, they had been in culture only 15-17 days. This short time frame is important as the longer cells are in culture, the less efficient they are as nuclear donors. Positive clones from the “cell expansion” plates were also passaged to provide genomic DNA for downstream applications. Because G418-selected fetal fibroblasts often senesce before large quantities of genomic DNA can be obtained, genomic DNA from the 96-well expansion plate was isolated and used whole-genome amplification (REPLI-g, Qiagen) to provide DNA for Southern blot analysis.

DNA sequence analysis was used to confirm the proper targeting site (FIG. 3C). Furthermore, Southern blots with ATM− and Neo^{R}-specific probes were used to identify clones with a targeted ATM allele and that were free of random integration. Seven ATM+/− male cell lines and five ATM+/− female cell lines were identified to date that meet the above criteria—processing all of the PCR-positive cell lines was not necessary; however, those cells and DNA have been preserved, if needed. It is believed the quality and quantity of these cells are ideal for nuclear transfer, and they will be used to generate ATM+/− pigs. Gene targeting statistics are shown in Table 2.

TABLE 2
Summary of ATM targeting efficiency and SCNT activity

| | Gene | | Embryos | | |
|---|---|---|---|---|---|
| | targeting | Number of | per transfer | Pregnancy | Live pigs |
| | efficiency* | transfers | (average) | rate† | per litter |
| Male | 0.4% | 3 | 140 | 34% | 3 |
| Female | 0.6% | 10 | 150 | 30% | 4.7 |

*Gene targeting efficiency reported as percentage of G418^{R} clones that were properly targeted, as determined by PCR.
†Pregnancy rate refers to full-term gestation.

### Example 4

### Nuclear Transfer and Propagation

One ATM+/− male cell line was used for somatic cell nuclear transfer (SCNT) to produce live male ATM+/− offspring. The SCNT process is described, for example, in U.S. Pat. No. 7,989,675, and in U.S. patent application Ser. Nos. 13/368,312 and 13/624,967.

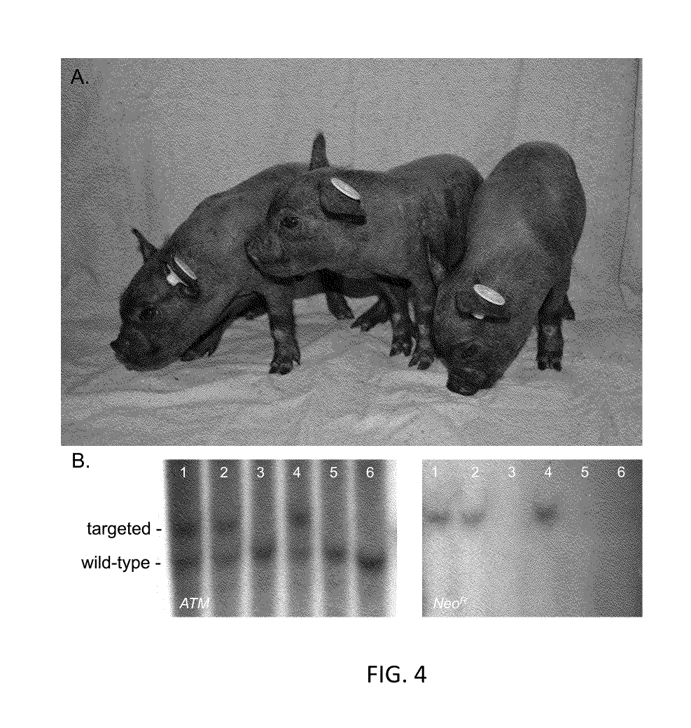

The resulting litter was born November 2011 and contained 5 piglets, 3 of which were ATM+/− (FIG. 4A). The two ATM+/+ piglets were the result of non-targeted cells evading the antibiotic selection and screening process. PCR genotyping and genomic Southern blots confirmed the ATM allele to be properly targeted (FIG. 4B), and was verified by DNA sequence analysis. Four female ATM+/− pigs were also produced in a similar manner.

Three litters of ATM+/− matings were delivered on Jan. 15, 2013, Jan. 29, 2013, and Feb. 24, 2013. These resulted in 2 ATM−/−, 5 ATM+/−, and 2 ATM+/+ piglets.

### Other Embodiments

Unless otherwise defined herein, all technical and scientific terms used herein have the ordinary meaning as commonly understood by one of ordinary skill in the art to which this invention pertains. Although methods and materials similar or equivalent to those described herein can be used in the practice or testing of the present invention, suitable methods and materials are described herein.

Although the invention has been described above in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

Other embodiments are within the following claims.

What is claimed is:

1-70. (canceled)

71. A transgenic, animal that models a human disease or condition comprising a large, non-human transgenic animal comprising an Ataxia-Telangiectasia (A-T) mutation of an endogenous Ataxia-Telangiectasia gene (ATM) and wherein the A-T mutation results in an altered expression of an ATM translation product and/or in expression of an ATM protein that corresponds to an alteration of a human A-T gene associated with a human disease or condition.

72. The transgenic animal of claim 71, wherein said transgenic animal is an ungulate.

73. The transgenic animal of claim 72, wherein said ungulate is selected from the group consisting of a swine, a cows-, a sheep, and a goats-.

74. The transgenic animal of claim 73, wherein said ungulate is a swine.

75. The transgenic animal of claim 71, wherein said A-T mutation is a deletion or a disruption of both ATM alleles.

76. The transgenic animal of claim 71, wherein said transgenic animal exhibits a phenotype and/or at least one symptom associated with the human disease, Ataxia-Telangiectasia.

77. The transgenic animal of claim 76, wherein said phenotype is present in a tissue of the transgenic animal, wherein said tissue is selected from the group consisting of an immune system tissue, a cerebellum, an eye, or a lung of said transgenic animal, and/or said at least one symptom is selected from the group consisting of a predisposition to cancer, a hypersensitivity to ionizing radiation, an immune system irregularity, a chromosomal instability, insulin-resistant diabetes, and a reduced or absent thymus.

78. The transgenic animal of claim 71, wherein said A-T mutation is in or at exon 57 of the endogenous ATM gene.

79. The transgenic animal of claim 71, wherein said A-T mutation is selected from the group consisting of:
i) a mutation affecting the production of the ATM translation product;
ii) a mutation resulting in the ATM translation product having an altered or an abnormal ATM protein function as compared to a wild-type ATM translation product; and
iii) a mutation resulting in the ATM translation product having an altered or an abnormal biological activity as compared to a wild-type translation product.

80. The transgenic animal of claim 79, wherein said A-T mutation is present in both alleles of the endogenous ATM gene.

81. The transgenic animal of claim 80, wherein both alleles of the ATM gene have the same mutation.

82. The transgenic animal of claim 71, wherein the A-T mutation results in a full or a partial inactivation of the ATM gene.

83. The transgenic animal of claim 71, wherein the A-T mutation comprises an insertion of an exogenous nucleic acid molecule.

84. The transgenic animal of claim 71, wherein the A-T mutation includes an early transcription termination sequence.

85. The transgenic animal of claim 83, wherein said A-T mutation occurs in or at exon 57.

86. The transgenic animal of claim 71, further comprising a stably integrated transgenic copy of a wild-type or a mutated exogenous nucleic acid sequence originally derived from a different animal.

87. An isolated cell line derived from the transgenic animal of claim 71.

88. An isolated cell line derived from the transgenic animal of claim 79.

89. An isolated cell line derived from the transgenic animal of claim 86.

90. The transgenic animal model of claim 71 wherein the human disease or condition is a telangiectasia, poor coordination, or a small, dilated blood vessel.

91. The transgenic animal model of claim 71 wherein said A-T mutation comprises SEQ ID NO: 1.

92. The transgenic animal model of claim 71 wherein said transgenic animal is obtained by a method comprising use of a cre/lox system.

93. The transgenic animal model of claim 71 wherein said transgenic animal is a selectable marker-free transgenic animal.

94. The transgenic animal model of claim 71 wherein said ATM mutation comprises an engineered termination stop codon.
